(19) United States
(12) Patent Application Publication
(10) Pub. No.: US 2012/0109201 A1
(43) Pub. Date: May 3, 2012

(54) INTERSEGMENTAL MOTION PRESERVATION SYSTEM FOR USE IN THE SPINE AND METHODS FOR USE THEREOF

(75) Inventors: Ryan M. Kretzer, Baltimore, MD (US); Bryan W. Cunningham, Baltimore, MD (US); Jeffrey Gordon, Baltimore, MD (US)

(73) Assignees: NEURAXIS LLC, Baltimore, MD (US); THE JOHNS HOPKINS UNIVERSITY, Baltimore, MD (US)

(21) Appl. No.: 13/098,365

(22) Filed: Apr. 29, 2011

Related U.S. Application Data
(60) Provisional application No. 61/329,865, filed on Apr. 30, 2010.
(60) Provisional application No. 61/386,229, filed on Sep. 24, 2010.
(60) Provisional application No. 61/430,140, filed on Jan. 5, 2011.

(51) Int. Cl. A61B 17/70; A61B 17/88

(52) U.S. Cl. 606/248; 606/279

(57) ABSTRACT

Medical devices and kits for dynamically stabilizing or preserving motion in a spine and limiting adjacent, non-adjacent, or isolated segment degeneration of the spine while providing a controlled, determinable range of motion at the treated site, as well as methods for surgical use of the system.

### RELATED APPLICATIONS

This application claims the benefit of prior U.S. Provisional Application No. 61/329,865, filed Apr. 30, 2010, U.S. Provisional Application No. 61/386,229, filed Sep. 24, 2010, and U.S. Provisional Application No. 61/430,140, filed Jan. 5, 2011. The content of each of these three U.S. Provisional Applications is incorporated herein by this reference.

### FIELD OF THE INVENTION

The invention relates to devices and methods for use in treating spinal degeneration. More particularly, the invention relates to devices for use in stabilization and preservation of motion within a degenerated spinal column.

### BACKGROUND OF THE INVENTION

Pedicle screw instrumentation in the spine has gained prominence in recent years due to the superior biomechanical properties provided by three column fixation. While these biomechanical advantages have improved construct stability over the operative spinal segment, the same factors that contribute to motion reduction also have been implicated in the progression of adjacent segment degeneration. Such adjacent segment degeneration is especially true with increasing construct length and when mild degenerative changes already exist at the supra- or infra-adjacent segments.

Efforts to address adjacent segment degeneration have included use of devices employing a pedicle screw-based design, with a rod or cord disposed in between the pedicle screw fixation points. One problem with this design has been that motion is decreased at the desired segment in a non-physiologic manner, which makes these devices prone to failure.

### SUMMARY OF THE INVENTION

The presently disclosed subject matter provides a posterior-based intersegmental motion preservation system for use in the spine. The system includes, in all embodiments, elastic elements which stabilize the area of the spine treated while preserving a desirable range of motion, preferably a substantially physiologic range of motion.

According to one aspect, the system provides a device comprising more than one elastic tensile member disposed opposite one another in the spinal region to be treated via attachment to either adjacent spinous processes or directly from a spinal instrumentation construct (e.g., crosslink, rod, pedicle screw, laminar screw, lateral mass screw, etc.) to an adjacent spinous process(es).

In a further aspect, the intersegmental motion preservation system provides a device which comprises a single elastic tensile member in the spinal region to be treated via attachment to either an adjacent spinous process or directly from a spinal instrumentation construct (e,g, crosslink, rod, pedicle screw, laminar screw, lateral mass screw, etc.) to an adjacent spinous process.

In particularly preferred embodiments, the elastic tensile members are elastic and provide a graded resistance to spinal motion by physiologically stiffening the posterior ligamentous complex. Advantageously, the range of motion provided by use of the invention is greater than achievable by spinal fusion up to the clinically indicated limit for the patient treated (e.g., a patient whose spinal column has been treated or damaged at a different site may need a more limited range of motion to prevent further injury than one whose only impairment is treated by use of the invention). Most advantageously, the range of motion provided by use of the invention is substantially physiologic compared to spinal fusion.

The device of the invention also can be implanted in a patient in a quick and efficient manner through a minimally invasive approach, thereby limiting further destabilization of the adjacent segment. In these ways, the invention provides a crucial tool for a spine surgeon to limit adjacent segment or non-adjacent segment range of motion and potential degeneration following operative fixation at all levels of the spine while providing for a substantially physiologic range of motion around the treated area.

In those respects, the invention also provides methods for (i) stabilizing adjacent bones; (ii) connecting adjacent vertebral levels; and/or (iii) treating kyphosis, e.g., proximal or distal junction kyphosis, or adjacent segment/non-adjacent segment degeneration (disc/facet degeneration or listhesis) in a subject in need of treatment thereof, through delivery of the system of the invention to the subject's spine.

To those ends, the one or more elastic tensile members of the devices of the inventive system stretch on application of tensile force generated by flexion, axial rotation, or lateral bending of a subject's spine around the treated region then return to their original configuration on release of the applied force. The tension on the connecting members and their stiffness can be varied as necessary to stabilize the spine without allowing it more than a range of motion advisable for the condition of the treated region.

To enable ready use of the invention in treating spinal degeneration, the system of the invention is preferably provided as a surgical kit including a device as disclosed herein, optionally including a selection of elastic members for use in patients of differing sizes and in a variety of conditions, tools for use in implantation of the device, and user instructions for reference by the surgeon.

Certain aspects of the presently disclosed subject matter having been stated herein above, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1 is a schematic showing a posterolateral view of one embodiment of the presently disclosed device comprising a pair of opposing elastic members 100 and 100′ which consist of bands each having a first and second end disposed in the same plane for attachment to a spinous process 10;

FIG. 2 is the device shown in FIG. 1 where the elastic members 100 and 100′ have been stretched by separation of spinous processes 10;

FIG. 3 is a schematic showing an exploded view of the device of FIG. 1 showing a pair of articulating attachment elements comprised of male connector 154 for interlocking joiner to female connector 160 through bores 12 in spinous processes 10;

FIG. 4 is a schematic showing a posterolateral view of one embodiment of the presently disclosed device comprising a pair of opposing connecting members 114 and 114′ which consist of bands, each having a first and second end disposed in the same plane for attachment to a spinous process 10 between which is an undulating, deformable surface;

FIG. 5 is the device shown in FIG. 4 where the elastic members 114 and 114′ have been stretched by separation of spinous processes 10;

FIG. 6 is a schematic showing an exploded view of the device of FIG. 4

FIG. 7a is a schematic showing a posterolateral view of one embodiment of the presently disclosed device comprising an elastic member 200 which contains with it a cord which acts as a stiffener and/or displacement limiter, and which has a first and second end disposed in the same plane for attachment to a spinous process 10;

FIG. 7b is a schematic similar to FIG. 7a, which shows elastic member 200 in a stretched configuration, thereby showing the action of cord 204;

FIG. 8a is a schematic showing a posterolateral view of one embodiment of the presently disclosed device comprising an elastic member 300 which consists of a spring having a first and second end disposed in the same plane for attachment to a spinous process 10;

FIG. 8b is a schematic similar to FIG. 8a, which shows elastic member 300 in a stretched configuration;

FIG. 9 is a schematic showing a posterolateral view of one embodiment of the presently disclosed device comprising a pair of opposing elastic members 400 and 400′ which comprise a pair of flattened, elastic tubes, each with a first and second end disposed in the same plane for attachment to a spinous process 10;

FIG. 10 is a schematic showing a posterolateral view of one embodiment of the presently disclosed device comprising a pair of opposing elastic members 500 and 500′ which consist of a pair of elastic cords, each having a first and second end disposed in the same plane for attachment to a spinous process 10;

FIG. 11 is a schematic showing a posterolateral view of one embodiment of the presently disclosed device comprising a pair of opposing elastic members 600 and 600′ which consist of a pair of tethers, each having a first and second end disposed in the same plane for attachment to a spinous process 10;

FIG. 12 is a schematic showing a posterolateral view of one embodiment of the presently disclosed device comprising a pair of opposing elastic members 100 and 100′ each having means for attachment to more than two spinous processes 10;

FIG. 13 is a schematic showing a posterolateral view of one embodiment of the male/female connector attachment mechanism of the disclosed device used to attach the elastic members to one or more spinous processes 10;

FIG. 14 is a schematic showing one embodiment of the presently disclosed device comprising an elastic member 650 attached to a pair of hooks 656 for attachment to a patient's spine.

FIG. 15 is a schematic showing a posterior view of three vertebrae and showing one embodiment of the presently disclosed device in which a pair of elastic members 702 and 702′ are attached to rods 700 and 700′ and to a spinous process 10;

FIG. 16 is a schematic showing a posterior view of three vertebrae and showing one embodiment of the presently disclosed device in which an elastic member 703 is attached to a crosslink 706 and to a spinous process 10;

FIG. 17 is a schematic showing a posterior view of one embodiment of the presently disclosed device in which a pair of elastic members 800 and 800′ is attached to a pair of spinous processes 10 with a pair of clamps 820;

FIG. 18 is an exploded view of the alternative embodiment of the invention shown in FIG. 17;

FIG. 19 is a schematic showing a posterolateral view of an alternative embodiment of the invention wherein a single elastic member 100 is attached to the spinous processes 10; and

FIG. 20 is an exploded view of the alternative embodiment of the invention shown in FIG. 19.

### DETAILED DESCRIPTION OF THE PREFERRED EMBODIMENTS

The presently disclosed subject matter now will be described more fully hereinafter with reference to the accompanying Figures, in which some, but not all embodiments of the presently disclosed subject matters are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

All publications and other printed materials referenced herein are incorporated herein by this reference.

### General Features of the Devices of the Invention

In preferred embodiments, the device of the invention provides a graded resistance to spinal motion by physiologically stiffening or replacing the posterior ligamentous complex with an intersegmental motion preservation system including one or more elastic tensile members. More particularly, the presently disclosed device and methods may be used to stiffen the spinal segment in a physiologic manner by recapitulating the supraspinous/interspinous ligament and ligamentum flavum complex that exists in vivo, or to replace part or all of the ligamentous complex. To this end, the device provides a determinable graded tensile resistance during spinal that responds to the force applied to a connection element of the device, which resumes its baseline shape once the applied load is removed. Advantageously, the range of motion provided by use of the invention is greater than achievable by spinal fusion up to the clinically indicated limit for the patient treated (e.g., a patient whose spinal column has been treated or damaged at a different site may need a more limited range of motion to prevent further injury than one whose only impairment is treated by use of the invention). Most advantageously, the range of motion provided by use of the invention is substantially physiologic compared to spinal fusion.

Another feature of the presently disclosed device is that it can be implanted through either a minimally invasive or open approach and does not require operative dissection over the facet joints or disruption of the supraspinous/interspinous ligament complex. The one or more bands of the device also provide a physiologic stiffening of the posterior tension band of the spine in flexion, axial rotation, and lateral bending loading that directly counteracts the most common failure modes of spinal segment degeneration (kyphosis or listhesis due to hypermobility).

In those embodiments of the inventive device which employ one or more elastic tensile members, as described in more detail herein below, the tensile member may each be a band, spring, tube, rod or similar structure, provided in a variety of lengths depending on patient size and region of the spine. Once implanted between the spinous processes or between a spinal instrumentation construct (e.g. crosslink, rod, pedicle screw, laminar screw, lateral mass screw, etc.) and an adjacent spinous process, the one or more bands elongate under application of physiological loads and then resume their baseline shape once the load is removed.

The elongation capacity of each elastic tensile member can be optimized by varying its geometry and/or stiffness; i.e., from a low stiffness providing significant flexibility allowing for spinal motion to substantially physiologic degrees, to a high stiffness allowing for little or no motion around the treatment site.

In one embodiment, each elastic tensile member is secured to adjacent spinous processes at the site of treatment by a pair of connection elements provided in, through, over, or around the spinous processes at opposing ends of each elastic tensile member. Alternatively, one end of the elastic tensile member can be attached directly to spinal instrumentation (e.g. crosslink, rod, pedicle screw, laminar screw, lateral mass screw, etc.) while the other end of the elastic tensile member attaches to an adjacent spinous process. The connection elements can be, for example, articulating male and female pairs formed of a biocompatible metal or rigid polymer (such as polyether ether ketone [PEEK] to avoid MRI artifact at both ends). The connection elements may also have a coating, such as hydroxyapatite or other material or surface modification, to improve osteointegration or adherence to the spinous process. The male connector preferably has a head or base to retain the connection element, to provide a bone in growth surface, and to provide a surface for interaction with a surgical instrument, such as a wrench or inserter. Each connection element pair is provided in a variety of lengths to fit the dimensions of the treatment site (e.g., individual spinous process width).

In one aspect, a retaining ring non-rotatably secures the elastic tensile member(s) to the spinous process. Sliding connections such as cotter pins, lynch pins, or clips could also be used. In a further aspect, however, the male connector and female connector can be made rotatably connected, such as a threaded connection or a spiral locking ring. In yet a further embodiment, a single connecting member may be employed for attachment of the elastic tensile member, such as a cotter pin, suture, cable, wire, or an open ring that would then be crimped closed.

For implantation of the device at a spinous process, a hole may be made through a spinous process using a drill, awl, or other mechanism. The width of the spinous process is measured to choose the appropriate length connector that will lock securely without extending excessively beyond the spinous process. The distance between adjacent spinous processes (if more than one is used to anchor the device) or, for anchoring to implanted spinal instrumentation (e.g. a crosslink, rod, pedicle screw, laminar screw, lateral mass screw, etc.) and an adjacent spinous process, is measured at neutral position. The measurement is used to select the appropriately sized elastic tensile members, which members are chosen to provide a suitable stiffness as herein described.

The elastic tensile members are then attached to one or more spinous processes and subsequently locked in place. Once locked, the elastic tensile member(s) and connecting member(s) combine to form the final device, supporting and stiffening the supraspinous/interspinous ligament complex without disrupting the posterior ligamentous complex. In cases of laminectomy and/or resection of the posterior ligamentous structures (supraspinous ligament and/or interspinous ligament and/or ligamentum fluvum and/or facet capsular ligaments), the device will serve to reconstruct the posterior ligamentous complex by attachment to the adjacent level spinous process(es) and spinal instrumentation (e.g. crosslink, rod, pedicle screw, laminar screw, lateral mass screw, etc.). Finally, the incision is closed in standard fashion.

Depending on the impairment, the stiffness of the invention can be determined by choice of various lengths, widths, thicknesses or diameters as well as materials of the elastic members in order to allow anywhere from a low to high degree of tensile stiffness: ranging from 0.1 N/mm to 1,000 N/mm. For example, the tensile stiffness may be less than 1,000, 900, 800, 700, 600, 500, 400, 300, 200 or 100 N/mm. Generally, suitable materials for use in the invention will be biocompatible ones including: biocompatible polymers such as nylon, PEEK, silicone, urethanes, aramids, polyethylenes and polypropylenes, as well as metals such as titanium and its alloys, stainless steel and cobalt chrome alloys, composites like carbon fiber, combinations of the previously mentioned materials, such as carbon fiber reinforced PEEK and other materials with properties meeting the foregoing criteria will be known to or readily ascertainable by those of ordinary skill in the art. The materials selection is most preferably made to provide a range of motion that is substantially equivalent to physiologic (or slightly less, as clinical requirements demand). In general, materials at the lower range of tensile stiffness will provide a greater range of motion, while stiffer ones will provide for a more controlled and delimited range of motion.

The device can be placed either above or below an existing spinal construct, or can be placed at a non-adjacent level that shows degenerative changes and is worrisome for further progression of degeneration in the future. One of ordinary skill in the art would appreciate that although the presently disclosed device is suited for the supra- or infra-adjacent segment above or below a spinal construct to reduce the incidence of proximal/distal junction kyphosis (PJK/DJK) or adjacent segment degeneration, the device can be placed at any level in the spine, including above or below a spinal construct or in the absence of prior spinal instrumentation. In other embodiments, the presently disclosed device also could be placed at a non-adjacent segment that has early signs of degeneration to delay further progression of spinal disease.

### Representative Embodiments of the Devices of the Invention

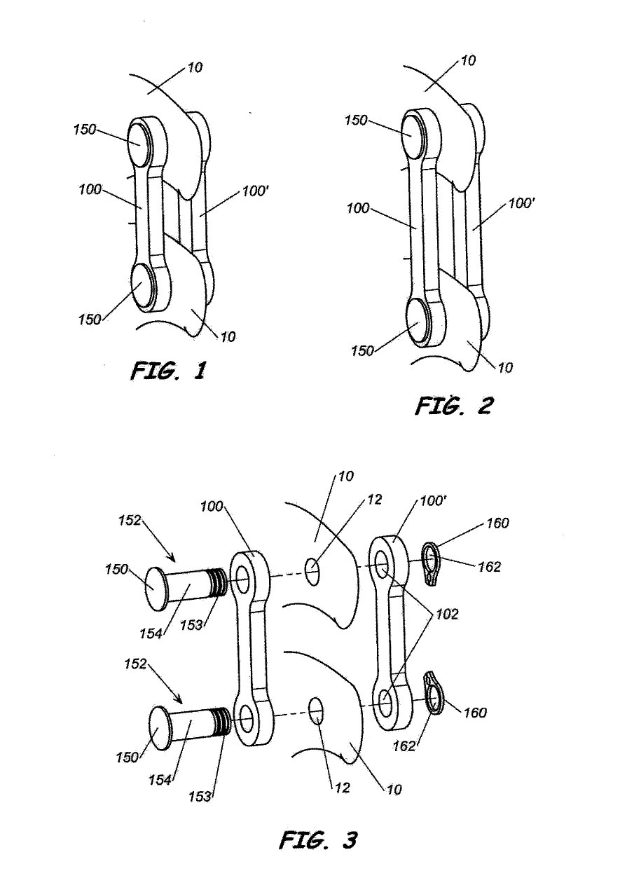

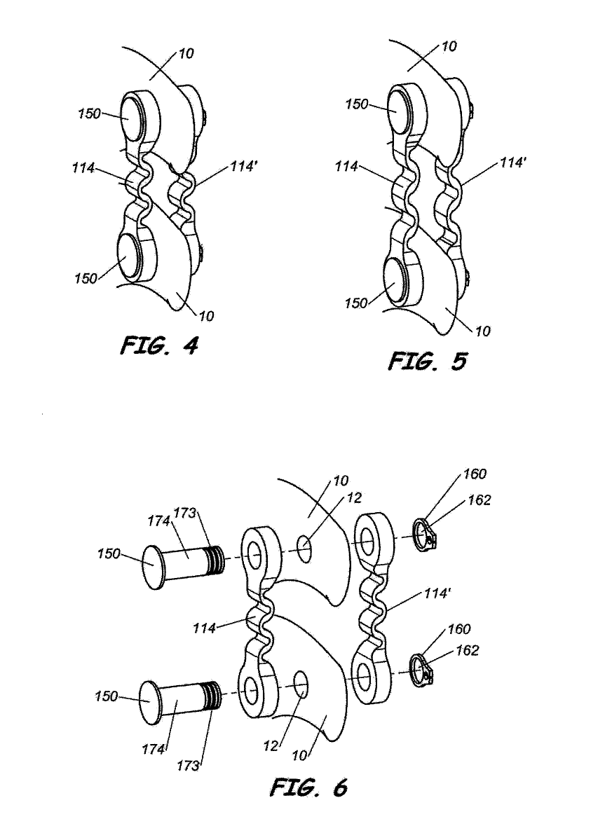

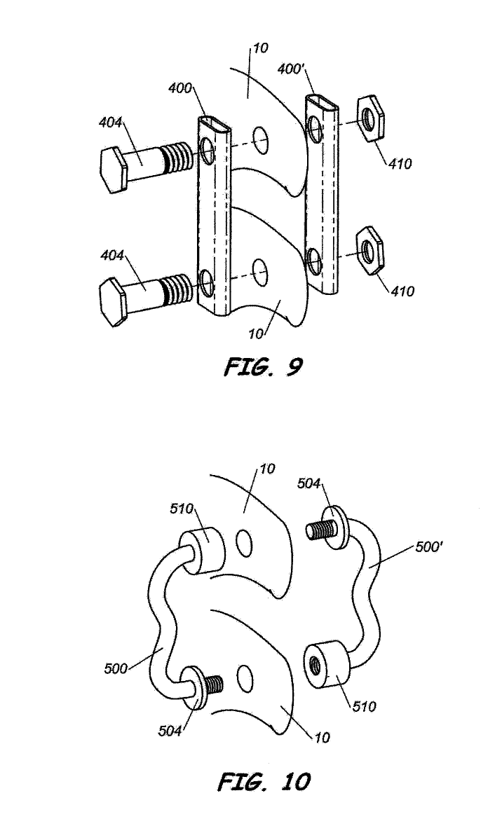

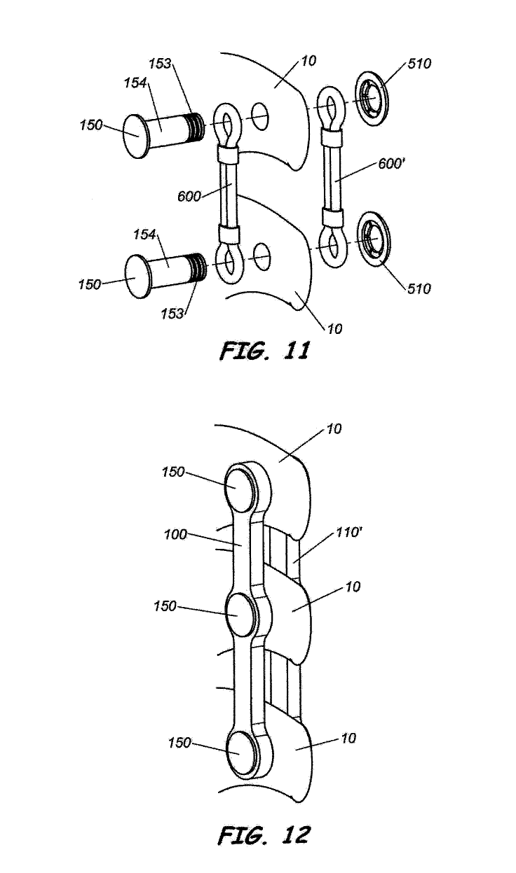

Referring now to FIGS. 1-3, one embodiment of the presently disclosed subject matter comprises two elastic members 100 and 100′, each having a first end and a second end for attachment to two spinous processes 10 by means of a male connector 152 and a corresponding female connector 160. As shown, elastic members 100 and 100′ are elastic bands, however the elastic members may be one or more other structures, such as a cord (FIGS. 10 & 11), a spring (FIGS. 8a & 8b), a band (FIGS. 4-6), a tether, a strap, a belt, a tube (FIG. 9), a wire, a tape, a cable, a suture, and the like. Referring to FIG. 3, male connector 152 and female connector 160 are complementary in that they can be articulately connected or united to form an interlocking joint. Male connector 152 comprises a head 150, a shaft 154, and one or more grooves 153. Shaft 154 is sized for insertion through a bore 162 of female connector 160, which is in turn sized to correspond in diameter to the bore 12 to be drilled or otherwise formed into spinous process 10.

Female connector 160 comprises bore 162, which is adapted to articulately connect, e.g., interlock, with one or more grooves 153 provided on shaft 154 of male connector 152. When interlocking elastic members 100 and 100′ are attached, joining of the male and female connectors by disposing bore 162 over a corresponding groove 153 in male adapter 152 creates a continuous connecting member on each side of the spinous process pair without disrupting the supraspinous/interspinous ligament complex. Connectors 152 and 160 are shown in the drawings as being generally cylindrical or round in shape. It will be understood however that other shapes may be utilized so long as the adapters can be connected through the bone.

An alternative configuration of a connecting member is shown in FIGS. 4-6. As shown, elastic members 114 and 114′ are undulating bands. The undulations provide the device additional capacity for motion in response to application of physiologic force, and allow elastic members 114 and 114′ to be manufactured from a relatively stiff biocompatible material such as titanium alloy or stainless steel. Elastic members 114 and 114′ would therefore elongate by flexion of the undulations much like leaf springs.

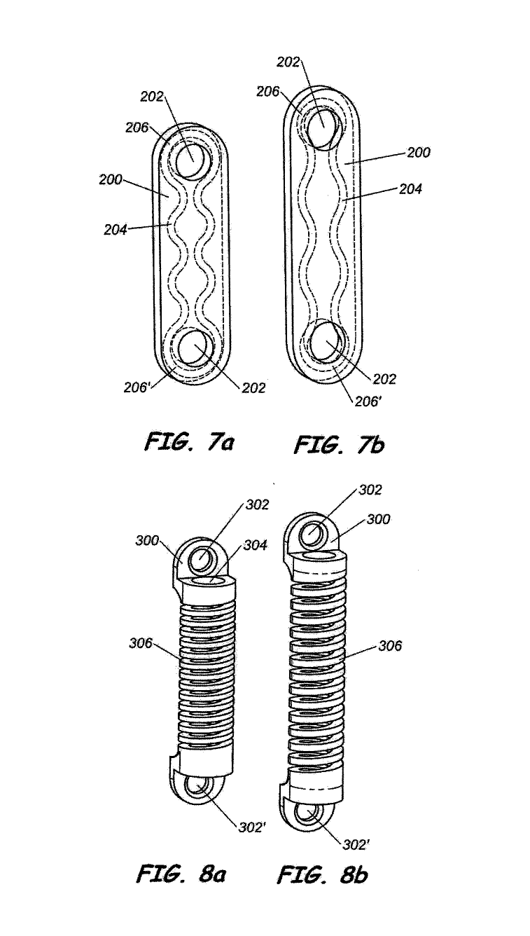

An additional alternative for use as connecting members in the device of the invention is shown in FIGS. 7a & 7b. As shown, band 204 is embedded within an elastic member 200. The function of elastic member 200 is much the same as described for elastic members 100 and 100′, with the addition of band 204 to stiffen the device or to act as a displacement limit for the device. FIG. 7b shows the same device as FIG. 7a, where the device has been stretched.

Another alternative for use as an elastic member in the device of the invention is depicted in FIGS. 8a & 8b. As shown, the elastic member can be an expandable helical spring 300 with a bore 304 to form a helical flexure 306. Such a spring may be produced, for example, either by helically bending a wire (not shown) or by machining a helical cut into a cylinder. Opposing open ends 302 of the expandable helical spring 300 facilitate connection to spinous processes 10. FIG. 8b shows the same device as FIG. 8a, where the device has been stretched.

Another alternative for use as an elastic member in the device of the invention is depicted in FIG. 9. As shown, elastic members 400 and 400′ are hollow, flattened tubes. The tube is elastically deformable with a degree of flexibility necessary to provide the desired clinical range of motion; e.g., substantially physiologic. A simple alternative adapter consisting of threaded male connector 404 and threaded female connector 410 can be used.

Another alternative for use as a connecting member in the device of the invention is depicted in FIG. 10. As shown, the elastic members are a substantially elastic pair of cords 500 and 500′. The cords can be connected by means of a threaded male end 504 and a correspondingly threaded female end 510, which may be crimped, swaged or otherwise attached to each cord to enable simple implantation and manufacture. Preferably, threaded female end 510 and threaded male end 504 are able to rotate without rotating cords 500 and 500′ to prevent cord twisting during insertion of male threaded end 504 into female threaded end 510.

Another alternative for use as a connecting member in the device of the invention is depicted in FIG. 11. As shown, the elastic members 600 and 600′ are elastic tethers, connectable by male adapter 154 and a retaining ring 510 which has tabs which engage into groove 153.

Another alternative for use as a connecting member in the device of the invention is depicted in FIG. 12. As shown, the connecting members 620 and 620′ are similar to connecting members 100 and 100′ described above except they connect more than two (in the illustrated case three) spinous processes 10.

Another alternative male connector 650 and female connector 620 are shown in FIG. 13. Male connector 650 incorporates a head 600 and a shaft 604 with a slot which forms two tines 610 and 610′. Female connector 620 incorporates a bore 605 and a recess 621. In operation, male connector 650 is guided into hole 12 in spinous process 10 until head 600 abuts spinous process 10. Female connector 620 is pushed onto the tip of male connector 650, thereby forcing together tines 610 and 610′ until they are able to pass through bore 605 at which point they return to their original positions and remain within recess 621.

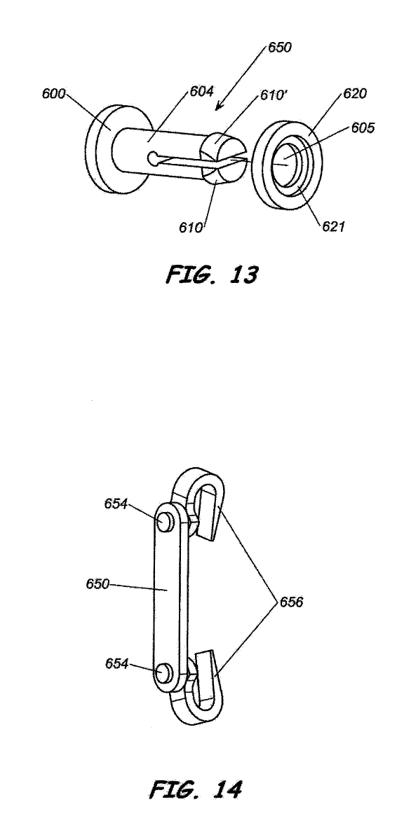

Another alternative embodiment of the invention is shown in FIG. 14. Elastic bridge member 650 is attached at each end to a hook 656 by attachment to a boss 654. Each of hooks 656 may be anchored to bone; e.g., by placement of a bone screw through each hook into a lamina or spinous process of a vertebra.

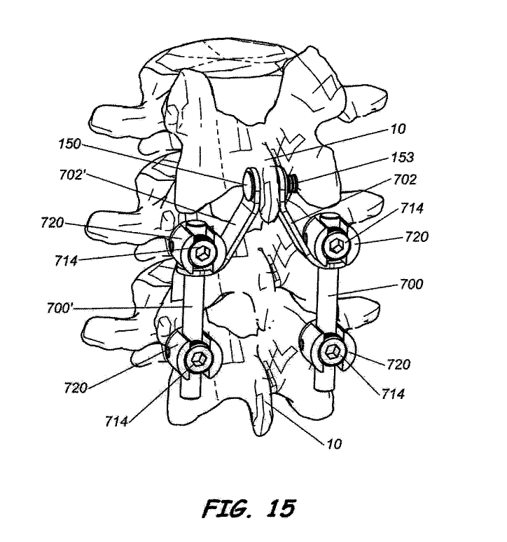

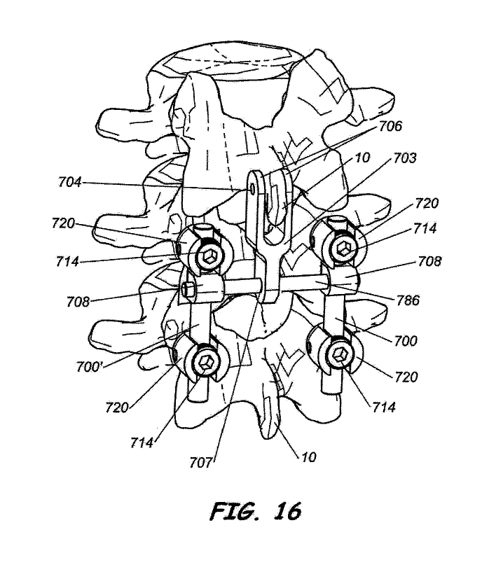

Another alternative embodiment of the invention is shown in FIG. 15. Here two vertebrae have been stabilized with rods 700 and 700′ by attachment to conventional pedicle screws 720 with connectors 714. Elastic elements 702 and 702′ are connected to rods 700 and 700′ and then connected to a spinous process 10 by means of a male connector 152 and female connector 160 in a fashion similar to the connectors discussed with respect to the embodiment of the invention shown in FIG. 3.

Another alternative embodiment of the invention is shown in FIG. 16. Here two vertebrae have been stabilized with rods 700 and 700′ by attachment to conventional pedicle screws 720 with connectors 714. Crosslink 786 is attached to rods 700 and 700′ with crosslink connectors 708, installed as familiar to those of ordinary skill in the art. Elastic member 703 extends from and is rotatably or non-rotatably connected to crosslink 786; for example, by sliding crosslink 786 through hole 707 in elastic member 703. Many other attachments can be easily envisioned to attach elastic element to crosslink 786 including, without limitation, applying washers to secure the attachment at the center of the crosslink. Opposite its connection to crosslink 786, elastic element 703 splits into tines 706, which are placed on either side of spinous process 10. The tines are secured to spinous process 10 via any suitable connection; e.g., a male/female connection such as male connector 152 and female connector 160 as described with respect to the embodiment of the invention shown in FIG. 3.

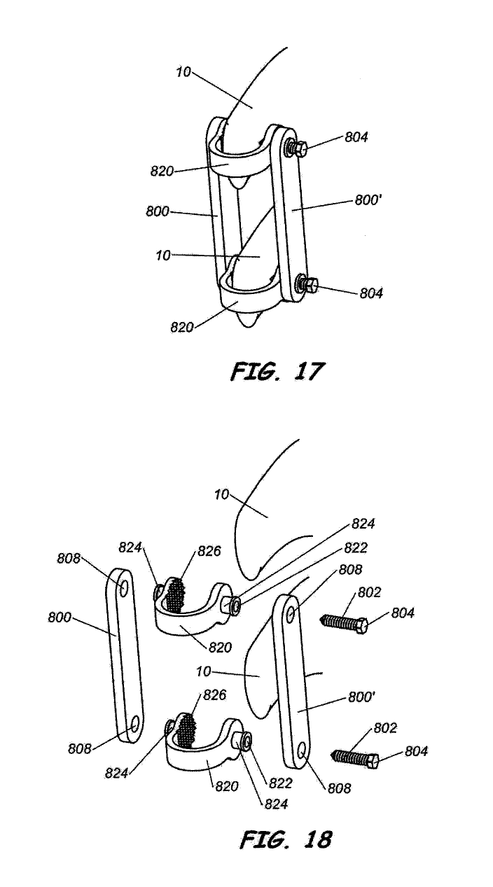

Another alternative embodiment of the invention is shown in FIGS. 17 and 18. Clamps 820 are comprised of bosses 824, knurled surfaces 826 and threaded holes 822 to accommodate attachment screw 802. Each clamp 820 is connected to a spinous process 10 by turning hex head 804 of attachment screw 802, thereby threading it into threaded hole 822 and constricting clamp 820 onto spinous process 10. Elastic members 800 and 800′ are connected to bosses 824 of clamps 820.

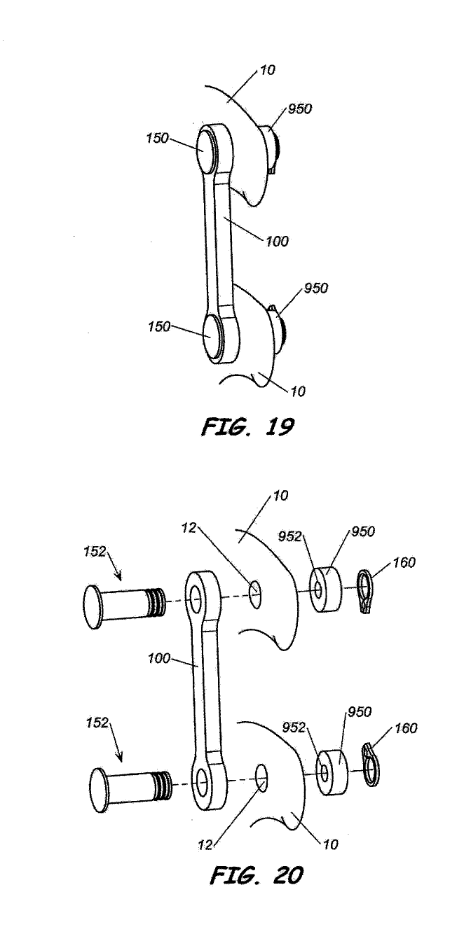

Another alternative embodiment of the invention employing a single elastic member is shown in FIGS. 19 and 20. Elastic member 100 is attached to spinous processes 10 by placement of male connector 152 through a hole in elastic member 100, a hole 12 in spinous process 10, a hole 952 in a washer 950 and a bore in female connector 160. Female connector 160 is attached to male connector 152 as described above. Washer 950 is optional, if needed to further stabilize the attachment of elastic member 100 to spinous process 10.

### Representative Methods for Use of the Devices of the Invention

Methods for implanting and deploying the devices of the invention include the following steps for use in (i) stabilizing adjacent bones; (ii) connecting adjacent vertebral levels; and/or (iii) preventing or treating kyphosis, listhesis, or segmental spinal instability in a subject in need of treatment thereof. In each such method, the steps comprise: forming two holes through adjacent spinous processes, attaching clamps to adjacent spinous processes, or forming one hole through an adjacent spinous process (or clamp) if connecting directly to spinal instrumentation (e.g. crosslink, rod, pedicle screw, laminar screw, lateral mass screw, etc.); connecting each end of a connecting member into the appropriate element of a connectable adapter (male or female element), wherein the connecting member is preferably pre-selected to provide a clinically appropriate level of stiffness; and joining the adapter elements through the spinous processes, or between one spinous process and an adjacent spinal instrumentation construct (e.g. crosslink, rod, pedicle screw, laminar screw, lateral mass screw, etc.), to secure the connecting members onto the spinous processes or onto adjacent spinal instrumentation.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The subject treated by the presently disclosed methods and devices in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

The terms “treat” or “treating,” and grammatical derivatives thereof, as used herein refer to any type of treatment that imparts a benefit to a subject afflicted with a disease or illness, including any measurable improvement in the condition of the subject (e.g., in one or more symptoms), reducing a symptom of the condition, inhibiting an underlying cause or mechanism related to the condition, delay in the progression of the condition, prevention or delay of the onset of the disease or illness, e.g., prophylactic treatment, enhancement of normal physiological functionality, and the like.

The term “effective,” as that term is used in the specification and/or claims, means adequate to accomplish a desired, expected, or intended result, e.g., to prevent, alleviate, or ameliorate symptoms of disease or prolong the survival of the subject being treated.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references are herein incorporated by reference to the same extent as if each publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A system for increasing the flexural stiffness between a treatment area consisting of adjacent spinal levels of a human spine, the system comprising:
a) one or more elastic tensile members having an elongate aspect for spanning the distance between a pair of adjacent spinous processes, the member comprising a first end opposing a second end and having a tensile stiffness between 0.1 N/mm and 1,000 N/mm; and
b) one or more connection elements for attachment of the one or more elastic tensile members to a spinous process, wherein each connection element is configured to be passed through both a bore in the first or second end of the one or more elastic tensile members and a bore in the spinous process,
wherein the stiffness of each elastic tensile member is selected to provide a clinically appropriate range of motion that is substantially physiologic around the treatment area as compared to spinal fusion.

2. The system of claim 1, wherein each of said one or more elastic tensile members is selected from the group consisting of a helical spring, a flexure, an elastic cord, an elastic plate, and an elastic tube.

3. The system of claim 1, wherein the connection element is selected from the group consisting of a bolt and nut, a grooved shaft and at least one retaining ring or clip, a wire, a cable, a suture, a cord, a shaft comprising a slot, and a hook.

4. The system of claim 1, wherein the connection element further comprises a washer or spacer on one or both opposing sides of the bore of the spinous process.

5. The system of claim 1, having a pair of elastic tensile members and a pair of connection elements, wherein the latter are configured to be passed through and span a bore in each of the first or second ends of the elastic tensile members and a bore in the spinous process, and wherein the elastic tensile members are positioned on opposing sides of the spinous process.

6. The system of claim 1, wherein the system further comprises a selection of elastic tensile members and user instructions for their selection and use by a physician.

7. A system for increasing the flexural stiffness between adjacent spinal levels of a human spine, the system comprising:
a) one or more elastic tensile members, each having an elongate aspect configured and dimensioned for spanning a spinous process and adjacent spinal instrumentation, each of the one or more members comprising a first end opposing a second end and having a tensile stiffness between 0.1 N/mm and 1,000 N/mm; and
b) one or more connection elements, wherein those for connection to a spinous process are configured to be passed through both a bore in the first or second end of the one or more elastic tensile members and a bore in the spinous process,
wherein the stiffness of each elastic tensile member is selected to provide a clinically appropriate range of motion that is substantially physiologic around the treatment area as compared to spinal fusion.

8. The system of claim 7, wherein each of said one or more elastic tensile members is selected from the group consisting of a helical spring, a flexure, an elastic cord, an elastic plate, and an elastic tube.

9. The system of claim 7, wherein the connection element is selected from the group consisting of a bolt and nut, a grooved shaft and at least one retaining ring or clip, a wire, a cable, a suture, a cord, a shaft comprising a slot, and a hook.

10. The system of claim 7, wherein the connection element further comprises a washer or spacer on one or both opposing sides of the bore of the spinous process.

11. The system of claim 7, wherein the system further comprises a selection of elastic tensile members and user instructions for their selection and use by a physician.

12. A system for increasing the flexural stiffness between adjacent spinal levels of a human spine, the system comprising:
a) one or more elastic tensile members having an elongate aspect, the member comprising a first end opposing a second end and having a tensile stiffness between 0.1 N/mm and 1,000 N/mm;
b) one or more connection elements for attachment of the one or more elastic tensile members to a spinous process, wherein the connection elements are adapted to be passed through both a bore in the first or second end of each of the one or more elastic tensile members and a bore in the spinous process;
c) one or more rigid rods configured for attachment to the spine; and
d) one or more further connection elements for connection of the one or more elastic tensile member to the one or more rigid rods,
wherein the stiffness of each elastic tensile member is selected to provide a clinically appropriate range of motion that is substantially physiologic around the treatment area as compared to spinal fusion.

13. The system of claim 12, wherein the connection element comprises a crosslink configured to connect the rigid rods and attachably engage the one or more elastic tensile member.

14. The system of claim 12, wherein the connection element comprises a bore through the one or more elastic tensile members, which bore is configured for passage therethrough of one of the rigid rods.

15. The system of claim 12, wherein the rigid rods are configured to couple with at least one pedicle screw, lateral mass screw, or laminar hook for attachment to the spine.

16. The system of claim 12, wherein the system further comprises a selection of elastic tensile members and user instructions for their selection and use by a physician.

17. A system for increasing the flexural stiffness between adjacent spinal levels of a human spine, the system comprising:
a) one or more elastic tensile members having an elongate aspect for spanning an adjacent spinous process, the members each comprising a first end opposing a second end and having a tensile stiffness between 0.1 N/mm and 1,000 N/mm; and
b) one or more connection means for attachment of an elastic tensile member to an adjacent spinous process, wherein the connection means is adapted to clamp onto the spinous process,
wherein the stiffness of each elastic tensile member is selected to provide a clinically appropriate range of motion that is substantially physiologic around the treatment area as compared to spinal fusion.

18. The system of claim 17, wherein the system further comprises a selection of elastic tensile members and user instructions for their selection and use by a physician.

19. A method for increasing the flexural stiffness between adjacent spinal levels of a spine in a human subject comprising surgically implanting the system of claim 1, 7, 12 or 17 into a chosen treatment site in the subject's spine.
